(19) United States
(12) Patent Application Publication
(10) Pub. No.: US 2015/0150814 A1
(43) Pub. Date: Jun. 4, 2015

(54) STABLE ORAL PHARMACEUTICAL COMPOSITION OF SOLIFENACIN

(71) Applicant: RANBAXY LABORATORIES LIMITED, New Delhi (IN)

(72) Inventors: Rajesh Srikrishan SHEAR, Gurgaon (IN); Girish MITTAL, Hisar (IN); Sumit MADAN, New Delhi (IN); Satish Kumar JAIN, Bilaspur (IN); Romi Barat SINGH, Varanasi (IN)

(21) Appl. No.: 14/556,604

(22) Filed: Dec. 1, 2014

(30) Foreign Application Priority Data
Nov. 29, 2013 (IN) 3481/DEL/2013

(51) Int. Cl. A61K 9/20; A61K 9/28; A61J 3/10; A61K 31/4725

(52) U.S. Cl. CPC A61K 9/2095; A61K 31/4725; A61K 9/2833; A61K 9/28; A61J 3/10

(57) ABSTRACT

The present invention relates to stable oral pharmaceutical compositions comprising solifenacin or a salt thereof prepared by a double compaction process, wherein the amorphous content of solifenacin or a salt thereof in the composition is not less than 80% by weight. It further relates to a method of treating or preventing overactive bladder syndrome by administering said pharmaceutical compositions.

### FIELD OF THE INVENTION

The present invention relates to stable oral pharmaceutical compositions comprising solifenacin or salts thereof prepared by a double compaction process, wherein the amorphous content of solifenacin or salts thereof in the composition is not less than 80% by weight. It further relates to a method of treating or preventing overactive bladder syndrome by administering said pharmaceutical compositions.

### BACKGROUND OF THE INVENTION

Solifenacin succinate is commercially available as film-coated tablets in 5 mg and 10 mg strengths. Chemically, solifenacin is (1S)-(3R)-1-azabicyclo [2.2.2] oct-3-yl3, 4-dihydro-1-phenyl-2(1H)iso-quinolinecarboxylate. Solifenacin succinate is a white to pale-yellowish-white crystal or crystalline powder and is freely soluble in water at room temperature.

Solifenacin is disclosed in U.S. Pat. Nos. 6,017,927 and 6,174,896.

PCT Publication No. WO 2005/092889 discloses a pharmaceutical composition comprising solifenacin or salts thereof wherein the amorphous content is within a range with no influence on product stability. The composition contains solifenacin or salts thereof and an inhibitor of the amorphous preparation and is preferably prepared by using a wet granulation process. It further discloses that if the amorphous content of solifenacin in the composition becomes greater than 77%, the amount of degradation product in the composition increases and exceeds 0.4% limit, and the composition becomes unstable.

PCT Publication No. WO 2006/070735 discloses a stable particulate pharmaceutical composition comprising solifenacin or salts thereof and a binder having a stabilizing action on solifenacin or salts thereof The binder stabilizes the drug by inhibiting retention of the amorphous form of solifenacin. It further discloses that the particulate composition is obtained by spraying a solution or suspension of solifenacin and a binder onto a core.

PCT Publication No. WO 2009/012987 discloses a stable pharmaceutical composition comprising solifenacin or a pharmaceutically acceptable salt thereof in amorphous form and a stabilizer. The amorphous solifenacin or salt thereof is stabilized by the stabilizer.

PCT Publication No. WO 2010/097243 discloses a dosage form comprising crystalline solifenacin or salts thereof and additives, wherein the composition is prepared in the absence of water or a liquid solvent. The water content of the dosage form is below 4% by weight based on the total weight of the dosage form.

The prior art teaches that the stability of pharmaceutical compositions comprising crystalline solifenacin or a salt thereof is affected if the content of amorphous solifenacin is higher.than 77% by weight of solifenacin in the composition. The inventors of the present application have found that solifenacin compositions remain stable despite having an amorphous content of more than 80% by weight of the drug, if prepared by a double compaction process.

### SUMMARY OF THE INVENTION

The present invention relates to stable oral pharmaceutical compositions comprising solifenacin or salts thereof prepared by a double compaction process, wherein the amorphous content of solifenacin or salts thereof in the composition is not less than 80% by weight. The pharmaceutical composition comprising solifenacin or salts thereof further comprises one or more pharmaceutically acceptable excipients selected from one or more of diluents, binders, disintegrants, lubricants, glidants, stabilizers, coloring agents, flavoring agents, sweeteners, film-forming agents, plasticizers, opacifiers, and mixtures thereof.

The present invention also includes a double compaction process for the preparation of said pharmaceutical composition and a method of treating or preventing overactive bladder syndrome by administering said pharmaceutical composition.

### DETAILED DESCRIPTION OF THE INVENTION

A first aspect of the present invention provides a stable oral pharmaceutical composition comprising solifenacin or salts thereof prepared by a double compaction process, wherein the amorphous content of solifenacin or salts thereof in the composition is not less than 80% by weight.

According to one embodiment of this aspect, the solifenacin salt is solifenacin succinate.

According to another embodiment of this aspect, solifenacin or a salt thereof is present in an amount of from about 0.1 mg to about 40 mg, more particularly from about 1 mg to about 15 mg.

According to another embodiment of this aspect, the stable oral pharmaceutical composition of the present invention further comprises one or more pharmaceutically acceptable excipients selected from one or more of diluents, binders, disintegrants, lubricants, glidants, coloring agents, flavoring agents, sweeteners, or mixtures thereof. The excipients may be used either intragranularly, extragranularly, or both.

According to another embodiment of this aspect, the oral pharmaceutical composition of the present invention is a tablet. In particular, it is a film-coated tablet.

According to another embodiment of this aspect, the oral pharmaceutical composition of the present invention is packed in a blister pack or in HDPE (high density polyethylene) bottles. The composition of the present invention is stored at a temperature below 27° C. and relative humidity of not more than 55%.

According to another embodiment of this aspect, the double compaction process comprises the steps of:
(a) blending solifenacin or a salt thereof and one or more intragranular excipients;
(b) compacting the blend of step (a) using a roller compactor;
(c) sifting and milling the compacts of step (b) to obtain granules;
(d) blending the granules of step (c) with one or more extragranular excipients;
(e) compressing the granules of step (d) into tablets; and
(f) optionally film coating the tablets of step (e).

A second aspect of the present invention provides a method of treating or preventing overactive bladder syndrome by administering a pharmaceutical composition comprising solifenacin or salts thereof prepared by a double compaction process, wherein the amorphous content of solifenacin or salts thereof in the composition is not less than 80% by weight.

According to one embodiment of this aspect, the pharmaceutical composition comprising solifenacin is administered in combination with other therapeutic agents.

The term “stable,” as used herein, refers to chemical stability, wherein not more than 2% w/w of total related substances are formed on storage at 25° C. and 60% relative humidity (RH) or at 40° C. and 75% relative humidity (RH) for a period of at least three months to the extent necessary for the sale and use of the composition.

The term “pharmaceutical compositions,” as used herein, refers to tablets or film-coated tablets. The tablets consist of a pre-determined quantity of active substance along with at least one pharmaceutically acceptable excipient.

The term “amorphous content of solifenacin,” as used herein, refers to the ratio of the amount of amorphous solifenacin or salts thereof to the total amount of amorphous and crystalline solifenacin or salts thereof in the pharmaceutical composition.

The term “about,” as used herein, refers to any value which lies within the range defined by a variation of up to ±10% of the value.

The term “solifenacin,” as used herein, refers to solifenacin and pharmaceutically acceptable salts thereof. The pharmaceutically acceptable salts of solifenacin include but are not limited to acid addition salts with a mineral acid such as hydrochloric acid, hydrobromic acid, hydroiodic acid, sulfuric acid, nitric acid, or phosphoric acid; or with an organic acid such as acetic acid, succinic acid, formic acid, propionic acid, oxalic acid, malonic acid, maleic acid, lactic acid, malic acid, citric acid, tartaric acid, carbonic acid, picric acid, fumaric acid, glutamic acid, methanesulfonic acid, ethanesulfonic acid, benzenesulfonic acid, etc. In particular the pharmaceutical composition of the present invention comprises solifenacin succinate. Solifenacin or salts thereof are present in the composition in an amount of about 0.1 mg to about 40 mg, in particular in an amount of about 1 mg to about 20 mg.

The solifenacin or salts thereof used for the preparation of the pharmaceutical composition of the present invention is present in crystalline form. During the manufacturing process, the crystalline form of solifenacin or salts thereof is converted to the amorphous form due to production steps such as compaction pressure, abrasion, and heat imposed during granulation. In the present invention, the amorphous content of the composition is maintained within a specified range at all times throughout the shelf life of the product. The amorphous content of solifenacin in the composition is not less than 80% by weight based on the total weight of solifenacin in the composition. The amount of amorphous solifenacin in the composition can be detected by measuring the amount of crystalline form of solifenacin in the composition by any of detection method known in the art, such as DSC, solid state NMR, infrared spectroscopy, raman spectroscopy, or X-ray diffraction, the latter being preferred.

Suitable diluents are selected from the group comprising lactose such as lactose anhydrous, lactose monohydrate, lactose DT (direct tableting), Flowlac®, and Pharmatose®; cellulose such as microcrystalline cellulose, co-processed microcrystalline cellulose, and powdered cellulose; starches such as pregelatinized starch, maize starch, rice starch, potato starch, and wheat starch; sugar alcohols such as mannitol, sorbitol, xylitol, and erythritol; dibasic calcium phosphate, dibasic calcium phosphate anhydrate; dibasic calcium phosphate dihydrate; tribasic calcium phosphate; calcium sulfate; calcium carbonate; and mixtures thereof. Particularly, the diluent is anhydrous lactose. The diluent is present in an amount of from about 50% to about 99% by weight, more particularly from about 60% to about 95% by weight of the composition.

Suitable binders are selected from the group comprising povidone, co-povidone, hydroxypropylmethyl cellulose, hydroxypropyl cellulose, hydroxyethyl cellulose, methyl cellulose, carboxymethyl cellulose sodium, xanthan gum, gum acacia, gum arabic, tragacanth, sorbitol, dextrose, sucrose, mannitol, gelatin, pullulan, sodium alginate, propylene glycol, polyvinyl alcohol, corn starch, maize starch, pregelatinized starch, methacrylates, carboxyvinyl polymers like carbomers, and mixtures thereof. Particularly, the binder is hydroxypropylmethyl cellulose. The binder is present in an amount of from about 0.1% to about 20% by weight, more particularly from about 1% to about 10% by weight of the composition.

Suitable disintegrants are selected from the group comprising hydroxypropyl cellulose, crospovidone, croscarmellose sodium, carboxymethyl cellulose sodium, carboxymethyl cellulose calcium, sodium starch glycolate, gums, alginic acid or alginates, starch, corn starch, modified starch, carboxymethyl starch, polyacrylates, and mixtures thereof. Particularly, the disintegrant is corn starch. The disintegrant is present in an amount of from about 0.1% to about 20% by weight, more particularly from about 1% to about 10% by weight of the composition.

Suitable lubricants are selected from the group comprising stearic acid, polyethylene glycol, magnesium stearate, calcium stearate, zinc stearate, talc, castor oil, sucrose esters of fatty acid, sodium stearyl fumarate, waxes, and mixtures thereof.

Suitable glidants or anti-sticking agents are selected from the group comprising talc, colloidal silicon dioxide, calcium silicate, magnesium silicate, silicon hydrogel, and mixtures thereof.

Suitable coloring agents and flavoring agents are selected from any FDA approved colors and flavors for oral use.

Suitable sweeteners are selected from the group comprising aspartame, saccharin sodium, acesulfame potassium, dried invert sugar, dextrose, glucose, fructose, galactose, levulose, maltose, neotame, sucralose, and mixtures thereof.

The pharmaceutical composition of the present invention may further be film coated using techniques known in the art, such as spray coating in a conventional coating pan or a fluidized bed processor, or dip coating. The film coat comprises film-forming polymers and one or more coating additives.

Suitable film-forming polymers are selected from the group comprising cellulose derivatives such as methylcellulose, hydroxymethyl cellulose, hydroxyethyl cellulose, hydroxypropyl cellulose, hydroxymethyl ethylcellulose, hydroxypropyl methylcellulose, sodium carboxymethyl cellulose, and ethyl cellulose; vinyl polymers such as polyvinylpyrrolidones; acrylic polymers; and mixtures thereof. Alternatively, commercially available coating compositions comprising film forming polymers marketed under various trade names, such as Opadry®, may be used for coating.

The coating additives comprise one or more of plasticizers, glidants or flow regulators, coloring agents, opacifiers, and lubricants.

Suitable plasticizers are selected from the group comprising castor oil, diacetylated monoglycerides, dibutyl sebacate, diethyl phthalate, glycerin, polyethylene glycols, propylene glycols, triacetin, triethyl citrate, and mixtures thereof. An opacifier like titanium dioxide may also be present in the coating.

Examples of solvents used for preparing the coating solution are selected from methyl alcohol, ethyl alcohol, isopropyl alcohol, n-butyl alcohol, acetone, acetonitrile, chloroform, methylene chloride, water, and mixtures thereof.

The composition of the present invention is used for treating or preventing conditions such as overactive bladder syndrome. The pharmaceutical composition of solifenacin or salts thereof may be administered in combination with other therapeutic agents used for treating or preventing overactive bladder or associated symptoms.

The present invention is illustrated below by reference to the following examples. However, one skilled in the art will appreciate that the specific methods are merely illustrative of the invention, and not to be construed as limiting its scope.

### EXAMPLES

### Example 1

| Ingredients | Quantity (% w/w) |
|---|---|
| Intragranular | |
| Solifenacin succinate | 3.244 |
| Anhydrous lactose | 86.294 |
| Hydroxypropylmethyl cellulose | 1.946 |
| Corn starch | 4.866 |
| Magnesium stearate | 0.487 |
| Extragranular | |
| Magnesium stearate | 0.487 |
| Film coating | |
| Ferric oxide (yellow) | 0.002 |
| Opadry ® white | 2.674 |
| Purified water | q.s. |

### Procedure

1. Solifenacin succinate, anhydrous lactose, hydroxypropylmethyl cellulose, and corn starch were blended in a blender, followed by lubrication with half the quantity of magnesium stearate.
2. The lubricated blend of step 1 was compacted using a roll compactor to obtain an 80:20 ratio of granules:fines.
3. The granules of step 2 were lubricated with the remaining magnesium stearate and the lubricated blend was compressed into tablets.
4. Ferric oxide along with purified water was milled.
5. Opadry° white was dispersed in the remaining purified water, followed by the addition of the material of step 4 to form a uniform dispersion.
6. The tablets of step 3 were coated with the Opadry® dispersion of step 5.

### Example 2

| Ingredients | Quantity (% w/w) |
|---|---|
| Intragranular | |
| Solifenacin succinate | 6.488 |
| Anhydrous lactose | 83.049 |
| Hydroxypropylmethyl cellulose | 1.946 |
| Corn starch | 4.866 |
| Magnesium stearate | 0.487 |
| Extragranular | |
| Magnesium stearate | 0.487 |
| Film coating | |
| Ferric oxide (red) | 0.002 |
| Opadry ® white | 2.675 |
| Purified water | q.s. |

### Procedure

1. Solifenacin succinate, anhydrous lactose, hydroxypropylmethyl cellulose, and corn starch were blended in a blender, followed by lubrication with half the quantity of magnesium stearate.
2. The lubricated blend of step 1 was compacted using a roll compactor to obtain an 80:20 ratio of granules:fines.
3. The granules of step 2 were lubricated with the remaining magnesium stearate and the lubricated blend was compressed into tablets.
4. Ferric oxide along with purified water was milled.
5. Opadry® white was dispersed in the remaining purified water, followed by the addition of the material of step 4 to form a uniform dispersion.
6. The tablets of step 3 were coated with the Opadry® dispersion of step 5.

The tablets prepared as per Example 2 were packed in blister packs and subjected to stability studies at 40° C./75% RH and 25° C./60% RH over a period of 6 months. The results are summarized in Table 1.

TABLE 1

| | 40° C. - 75% RH | | | 25° C. - 60% RH | | |
|---|---|---|---|---|---|---|
| Storage | | 3 | 6 | | 3 | 6 |
| conditions | Initial | months | months | Initial | months | months |
| Total Related | 0.03 | 0.36 | 0.24 | 0.03 | 0.36 | 0.13 |
| substances | | | | | | |
| (% w/w)* | | | | | | |
| % Amorphous | 90.7 | >80 | >80 | 90.7 | >80 | >80 |
| content | | | | | | |

*% Total Related substances should not be more than 2%.

From the above data, it is clear that the tablets prepared according to the present invention remain stable for 6 months and that the amorphous content of solifenacin succinate in the composition is not less than 80% by weight.

### Example 3

| Ingredients | Quantity (% w/w) |
|---|---|
| Intragranular | |
| Solifenacin succinate | 3.236 |
| Anhydrous lactose | 86.084 |
| Hydroxypropylmethyl cellulose | 1.942 |
| Corn starch | 4.854 |
| Magnesium stearate | 0.485 |
| Extragranular | |
| Magnesium stearate | 0.485 |
| Film coating | |
| Ferric oxide (yellow) | 0.001 |
| Opadry ® white | 2.912 |
| Purified water | q.s. |

### Procedure

1. Solifenacin succinate, anhydrous lactose, hydroxypropylmethyl cellulose, and corn starch were blended in a blender, followed by lubrication with half the quantity of magnesium stearate.
2. The lubricated blend of step 1 was compacted using a roll compactor to obtain an 80:20 ratio of granules:fines.
3. The granules of step 2 were lubricated with the remaining magnesium stearate and the lubricated blend was compressed into tablets.
4. Ferric oxide along with purified water was milled.
5. Opadry® white was dispersed in the remaining purified water, followed by the addition of the material of step 4 to form a uniform dispersion.
6. The tablets of step 3 were coated with the Opadry® dispersion of step 5.

The tablets prepared as per Example 3 were packed in blister packs and subjected to stability studies at 40° C./75% RH and 25° C./60% RH over a period of 6 months. The results are summarized in Table 2.

TABLE 2

| | 40° C. - 75% RH | | | 25° C. - 60% RH | | |
|---|---|---|---|---|---|---|
| Storage | | 3 | 6 | | 3 | 6 |
| conditions | Initial | months | months | Initial | months | months |
| Total Related | 0.13 | 0.41 | 0.39 | 0.13 | 0.21 | 0.26 |
| substances | | | | | | |
| (% w/w)* | | | | | | |
| % Amorphous | >80 | >80 | >80 | >80 | >80 | >80 |
| content | | | | | | |

*% Total Related substances should not be more than 2%.

From the above data, it is clear that the tablets prepared according to the present invention remain stable for 6 months and that the amorphous content of solifenacin succinate in the composition is not less than 80% by weight.

We claim:

1. A stable oral pharmaceutical composition comprising solifenacin or salts thereof prepared by a double compaction process, wherein the amorphous content of the solifenacin or salts thereof in the composition is not less than 80% by weight.

2. The stable oral pharmaceutical composition according to claim 1, wherein the solifenacin salt is solifenacin succinate.

3. The stable oral pharmaceutical composition according to claim 1, wherein the composition contains from about 0.1 mg to about 40 mg of the solifenacin or salts thereof

4. The stable oral pharmaceutical composition according to claim 1, wherein the composition further comprises one or more pharmaceutically acceptable excipients selected from diluents, binders, disintegrants, lubricants, glidants, coloring agents, flavoring agents, sweeteners, and mixtures thereof

5. The stable oral pharmaceutical composition according to claim 1, wherein the composition is a tablet.

6. The stable oral pharmaceutical composition according to claim 5, wherein the tablet is film-coated.

7. The stable oral pharmaceutical composition according to claim 6, wherein the film coat comprises film-forming polymers and one or more coating additives.

8. A double compaction process for preparing the stable oral pharmaceutical composition according to claim 1, wherein the processes comprises::
(a) blending solifenacin or a salt thereof and one or more intragranular excipients;
(b) compacting the blend of step (a) using a roller compactor;
(c) sifting and milling the compacts of step (b) to obtain granules;
(d) blending the granules of step (c) with one or more extragranular excipients;
(e) compressing the granules of step (d) into tablets; and
(f) optionally film coating the tablets of step (e).

9. A method of treating or preventing overactive bladder syndrome by administering the stable oral pharmaceutical composition according to claim 1.

10. The method of treatment according to claim 9, wherein the stable oral pharmaceutical composition is administered in combination with other therapeutic agents.
